(19) United States
(12) Patent Application Publication
(10) Pub. No.: US 2022/0280722 A1
(43) Pub. Date: Sep. 8, 2022

(54) DEVICES, SYSTEMS AND METHODS FOR PREDICTING FUTURE PHARMACOKINETIC PARAMETERS FOR A PATIENT UTILIZING INPUTS OBTAINED FROM AN ELECTROCHEMICAL SENSOR

(71) Applicants: THE REGENTS OF THE UNIVERSITY OF CALIFORNIA, Oakland, CA (US); UNIVERSITY OF UTAH RESEARCH FOUNDATION, Salt Lake City, UT (US)

(72) Inventors: Preetham Suresh, San Diego, CA (US); Jerry Ingrande, Jamul, CA (US); Joseph Wang, San Diego, CA (US); Ken B. Johnson, Cottonwood Heights, UT (US); Talmage Egan, Salt Lake City, UT (US)

(21) Appl. No.: 17/688,699

(22) Filed: Mar. 7, 2022

Related U.S. Application Data
(60) Provisional application No. 63/157,566, filed on Mar. 5, 2021.

(51) Int. Cl. A61M 5/172; G16H 20/17; G16H 70/40

(52) U.S. Cl. CPC A61M 5/1723; G16H 20/17; G16H 70/40; A61M 2202/048; A61M 2230/20

(57) ABSTRACT

Systems and methods are provided for combining predictive analytics with a cutting-edge electrochemical sensor having specialized coatings designed to reduce biofouling to (1) monitor drug concentration data of a patient in real-time; and (2) predict future pharmacokinetic parameters for the patient more accurately than existing technologies. Embodiments may construct highly accurate and patient-specific pharmacokinetic models which can dynamically adjust predictions of future pharmacokinetic parameters as they receive real-time drug concentration data from the electrochemical sensor. Certain embodiments may automatically adjust administration of a drug to a patient based on the aforementioned predictions and pharmacokinetic models. Other embodiments may provide a notification to a clinician containing, e.g., a recommended course of drug administration before a patient is woken up.

### REFERENCE TO RELATED APPLICATION

The present application claims priority to U.S. Provisional Patent Application No. 63/157,566, filed Mar. 5, 2021 and titled “Devices, Systems and Methods for Training an Infusion Pump By Measured Plasma Concentration,” which is incorporated herein by reference in its entirety.

### TECHNICAL FIELD

The present disclosure relates generally to medical technologies, and more particularly, some embodiments relate to monitoring and predicting drug concentrations in patients.

### DESCRIPTION OF RELATED ART

Hypnotic and analgesic drugs such as propofol (PPF) and fentanyl (FTN) may be administered intravenously to induce anesthesia in a patient. During surgical operations, clinicians try to maintain circulating concentrations of these drugs (i.e. in-vivo drug concentrations) within target ranges based on a personalized dosage for the patient. While an inadequate dose of anesthesia can result in problems such as pain and intraoperative awareness (i.e. wakefulness), overdosing of anesthetics may lead to respiratory distress/failure, and decreased blood pressure. These consequences can lead to morbidity and mortality.

### BRIEF DESCRIPTION OF THE DRAWINGS

The technology disclosed herein, in accordance with one or more various embodiments, is described with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is an example diagram depicting a generalized example of a 3-compartment model which can be used to describe drug concentration vs. time in a patient's blood/plasma, in accordance with various embodiments of the present disclosure.

FIG. 2 depicts two example diagrams illustrating drug concentration vs. time, in accordance with various embodiments of the present disclosure.

FIG. 3 illustrates an example iterative process performed by a computing system 300 for predicting future pharmacokinetic parameters for a patient utilizing real-time inputs obtained from a catheter-based electrochemical sensor, in accordance with various embodiments of the present disclosure.

FIG. 4 illustrates an example schematic illustration of dual PPF/FTN sensing on integrated microcatheter sensor, in accordance with various embodiments of the present disclosure.

FIG. 5 illustrates example detection of PPF/FTN at integrated dual microcatheter sensor in protein-rich artificial plasma medium, in accordance with various embodiments of the present disclosure.

FIG. 6 illustrates example selectivity investigation of the PVC/CP and PVC/CNT-CP sensors against various potential interferents, in accordance with various embodiments of the present disclosure.

FIG. 7 illustrates example SEM images, in accordance with embodiments of the present disclosure.

FIG. 8 illustrates example SEM characterizations of the fabrication steps, in accordance with embodiments of the present disclosure.

FIG. 9 illustrates example simultaneous PPF/FTN mixture analysis on integrated PVC/CP and PVC/CNT-CP microcatheter sensor, in accordance with various embodiments of the present disclosure.

FIG. 10 illustrates example Individual PPF/FTN detection in a whole blood medium or from a patient derived sample, in accordance with embodiments of the present disclosure.

FIG. 11 illustrates an example computing component that may be used to implement features of various embodiments of the disclosure.

The figures are not intended to be exhaustive or to limit the presently disclosed technology to the precise form disclosed. It should be understood that the presently disclosed technology can be practiced with modification and alteration, and that the disclosed technology be limited only by the claims and the equivalents thereof.

### DETAILED DESCRIPTION OF THE EMBODIMENTS

To maintain in-vivo drug concentrations within target ranges, current technologies utilize generic pharmacokinetic models to estimate theoretical in-vivo drug concentrations as a function of time (as used herein a pharmacokinetic model may refer to a mathematical model which correlates in-vivo drug concentration vs. time). These generic pharmacokinetic models are typically constructed using prospectively gathered blood/plasma concentration data from a population of patients. In particular, various regression techniques may be used to create a best fit curve for the population data. This generic pharmacokinetic model/best fit curve can be used to simulate in-vivo drug concentrations for a given patient as a function of time. Based on these simulated predictions, a clinician may adjust administration of anesthetic drugs to the patient as needed to maintain in-vivo concentration within target ranges.

Like untailored suits, these generic pharmacokinetic models do not fit all patients well. In other words, even within demographic population blocks (e.g. 25-30 year old men of similar heights and weights), pharmacokinetic parameters/processes may vary significantly from patient to patient, day to day, surgery to surgery, etc. Factors which can cause such deviation may include variability in body fat percentage, variability in rates of drug metabolism and elimination based on genetic differences, physiologic derangements such as hemorrhage, compartment volumes, etc.

A short-coming of the aforementioned generic pharmacokinetic models is their inability to respond in-real time to dynamic, patient-specific data. Instead, they rely on static, prospectively gathered (i.e. historical) population data which clinicians hope will approximate the actual pharmacokinetic process in their patient.

Generic pharmacokinetic models have been used ubiquitously because existing technologies have been unable to monitor a patient's pharmacokinetic parameters (e.g. in-vivo drug concentrations vs. time) in real-time in clinical settings like surgical operations. Existing technologies for detecting drugs such as PPF and FTN (e.g., liquid or gas chromatography in combination with mass spectrometry) involve time-consuming processes and bulky instruments. For example, these technologies often require drawing a blood sample, processing the blood sample in some way (e.g., centrifuging it) and then perform a time consuming assay using a machine that is bulky and not proximate to the point of care. Accordingly, these existing technologies are difficult to adapt into portable, miniaturized devices capable of real-time monitoring of dynamic drug concentrations in a patient.

Electrochemical technologies have shown promise as real-time monitors as they are highly sensitive, offer fast response times, and may be implemented using portable low-cost instrumentation. However, such electrochemical systems have had difficulty with long-time monitoring of drugs in the bloodstream due to reduced measurement accuracy over time due to variables such as biofouling (as used herein biofouling may refer to the degradation of an electrochemical sensor due to contact with biological material such as blood).

Against this backdrop, embodiments of the presently disclosed technology combine predictive analytics with a cutting-edge electrochemical sensor having specialized coatings designed to reduce biofouling to (1) monitor drug concentration in a patient in real-time; and (2) predict future pharmacokinetic parameters for the patient more accurately than existing technologies. Accordingly, embodiments may construct highly accurate and patient-specific pharmacokinetic models which can dynamically adjust predictions of future pharmacokinetic parameters as they receive data from the electrochemical sensor. Certain embodiments may automatically adjust administration of a drug to a patient based on the aforementioned predictions and pharmacokinetic models. Other embodiments may provide a notification to a clinician containing, e.g., a recommended cessation of a drug administration to guide timely emergence from anesthesia.

In various embodiments, Bayesian statistics may be used to predict future pharmacokinetic parameters for a patient. Bayesian statistical methods use Bayes' theorem to compute and update probabilities after obtaining new data. Bayes' theorem describes the conditional probability of an event based on data as well as prior information or beliefs about the event or conditions related to the event. Because Bayesian statistical methods/models are designed to update probabilities after obtaining new data, they are well-suited for making pharmacokinetic parameter predictions in accordance with embodiments of the presently disclosed technology. In other words, these Bayesian models may constantly adapt and improve their predictions in response to new patient-specific drug concentration data obtained from the aforementioned electrochemical sensor.

Referring again to the electrochemical sensor (sometimes referred to herein as a microcatheter sensor or microcatheter-based sensor), various embodiments provide a microcatheter-based sensor capable of continuous electrochemical monitoring of anesthetic drugs such as PPF and FTN using square-wave voltammetric detection. In various embodiments, this microcatheter-based sensor can monitor PPF and FTN simultaneously.

The microcatheter-based sensor may comprise a catheter tube (e.g., a Teflon-based tube) with internally disposed electrodes. For example, a first working electrode and a first reference electrode (e.g., an Ag/AgCl wire) may be disposed within the external catheter tube. The first working and reference electrodes may be used to detect PPF concentration in a patient. In certain embodiments, a second working electrode and a second reference electrode may be disposed within the catheter tube as well. The second working and reference electrodes may be used to detect FTN concentration in the patient.

As described above, the first and second working electrodes may be specially coated to reduce biofouling. For example, the first working electrode may be a carbon paste (CP) material coated with polyvinyl chloride (PVC). The second working electrode may be a carbon nanotube (CNT)-incorporated CP material coated with multiple material layers. These material layers may comprise a PVC material layer, an electrochemically reduced graphene oxide (erGO) material layer, and a gold (Au) nanoparticle layer. Such a multilayered design may be referred to as a PVC/erGO/Au/CNT-CP electrode.

As described above, specialized electrode coatings may reduce biofouling when the microcatheter-based sensor is inserted intravenously in a patient. By reducing biofouling, embodiments may improve detection accuracy and device longevity. Accordingly, embodiments may reduce long-held concerns that electrochemical systems are poorly-suited for continuous long-time monitoring of drugs in the bloodstream.

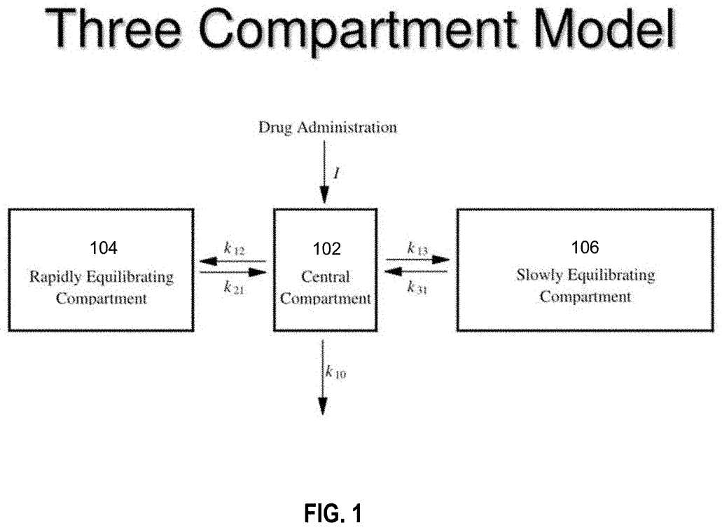

FIG. 1 is an example diagram depicting a generalized example of a 3-compartment model. Although there is no exact correspondence with anatomical parts, each compartment represents places in the body where drug concentration changes at similar rates. In particular, central compartment 102 may represent a patient's blood/plasma, rapidly equilibrating compartment 104 may represent a patient's rapidly equilibrating tissue(s) such as the liver and kidneys, and slowly equilibrating compartment 106 may represent a patient's slowly equilibrating tissue(s) such as bone and fatty tissue. Such a three-compartment model may be used as a basis for pharmacokinetic equations/models that describe drug concentrations vs. time in an individual patient.

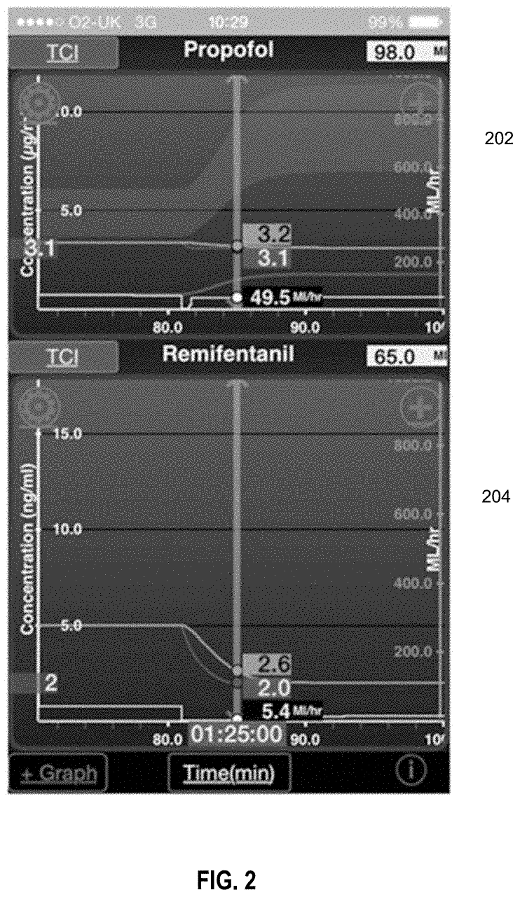

FIG. 2 depicts two example diagrams illustrating drug concentration vs. time. Diagram 202 illustrates concentration vs. time for propofol in an example patient, and diagram 204 illustrates concentration vs. time for remifentanil in the example patient. The vertical lines in each diagram denote the present. In accordance with embodiments of the present technology, the concentration vs. time curves to the left of the vertical line represent measured concentration values for the respective drugs in a patient's blood/plasma. The concentration vs. time curves to the right of the vertical line represent predicted concentration vs. time values. As alluded to above, Bayesian statistical techniques may be used to improve/refine pharmacokinetic models to better predict these future concentration vs. time values in an individual patient based on measurements obtained from the electrochemical sensor of the present disclosure.

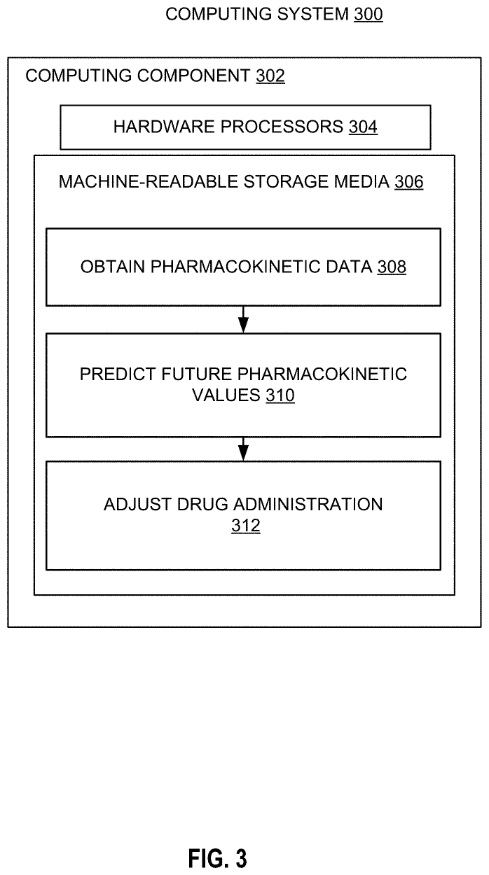

FIG. 3 illustrates an example iterative process performed by a computing system 300 for predicting future pharmacokinetic parameters for a patient utilizing real-time inputs obtained from a catheter-based electrochemical sensor, in accordance with various embodiments of the present disclosure. Computing system 300 may be comprised of one or more computing components, such as computing component 302. Computing component 302 may be, for example, a server computer, a controller, or any other similar computing component capable of processing data. In the example implementation of FIG. 3, the computing component 302 includes a hardware processor 304, and machine-readable storage medium 306.

Hardware processor 304 may be one or more central processing units (CPUs), semiconductor-based microprocessors, and/or other hardware devices suitable for retrieval and execution of instructions stored in machine-readable storage medium 306. Hardware processor 304 may fetch, decode, and execute instructions, such as instructions 308-312, to control processes or operations for optimizing the system during run-time. As an alternative or in addition to retrieving and executing instructions, hardware processor 304 may include one or more electronic circuits that include electronic components for performing the functionality of one or more instructions, such as a field programmable gate array (FPGA), application specific integrated circuit (ASIC), or other electronic circuits.

A machine-readable storage medium, such as machine-readable storage medium 306, may be any electronic, magnetic, optical, or other physical storage device that contains or stores executable instructions. Thus, machine-readable storage medium 306 may be, for example, Random Access Memory (RAM), non-volatile RAM (NVRAM), an Electrically Erasable Programmable Read-Only Memory (EEPROM), a storage device, an optical disc, and the like. In some examples, machine-readable storage medium 306 may be a non-transitory storage medium, where the term “non-transitory” does not encompass transitory propagating signals. As described in detail below, machine-readable storage medium 306 may be encoded with executable instructions, for example, instructions 308-312.

Hardware processor 304 may execute instruction 308 to obtain, from a catheter-based electrochemical sensor inserted in a patient, real-time drug concentration data associated with a first drug in the patient. In various embodiments, hardware processor 304 may execute instruction 308 to also obtain real-time drug concentration data associated with a second drug in the patient. In certain embodiments the first and second drugs may be anesthetic drugs such as propofol and fentanyl respectively.

The catheter-based electrochemical sensor may be any of the catheter-based electrochemical sensors described in the present disclosure. As described above, the catheter-based electrochemical sensor may use square-wave voltammetric detection to detect the drug concentration data.

In various embodiments, the catheter-based electrochemical sensor may comprise a catheter tube (e.g., a Teflon-based tube) with internally disposed electrodes. A first working electrode and a first reference electrode (e.g., an Ag/AgCl wire) may be disposed within the external catheter tube. The first working electrode and first reference electrode may be used to detect drug concentration data associated with the first drug in the patient. In certain embodiments, a second working electrode and a second reference electrode may be disposed within the catheter tube as well. The second working electrode and second reference electrode may be used to detect drug concentration data associated with the second drug in the patient.

The first and second working electrodes may be comprised of carbon paste (CP) materials. In some embodiments, the CP electrode materials can be modified to tune the sensitivity and dynamic range of the first and second drug and address challenges for realizing simultaneous monitoring.

In certain examples, the first working electrode may be a CP material coated with polyvinyl chloride (PVC). The second working electrode may be a carbon nanotube (CNT)-incorporated carbon paste material coated with multiple material layers. These material layers may comprise a PVC material layer, an electrochemically reduced graphene oxide (erGO) material layer, and a gold (Au) nanoparticle layer. Such a multilayered design may be referred to as a PVC/erGO/Au/CNT-CP electrode.

As described above, these specialized electrode coatings may reduce biofouling when the catheter-based electrochemical sensor is inserted intravenously in a patient. By reducing biofouling, embodiments may improve detection accuracy and device longevity. Accordingly, embodiments may reduce long-held concerns that electrochemical systems are poorly-suited for continuous long-time monitoring of drugs in the bloodstream.

The real-time drug concentration data associated with the first drug in the patient may refer to real-time (or close to real-time, e.g., within milliseconds) data associated with the administration of the first drug to the patient. For example, the real-time drug concentration data associated with the first drug may comprise the concentration of the first drug in the patient's plasma. The real-time drug concentration data associated with the second drug may be defined similarly.

Hardware processor 304 may execute instruction 310 to predict, based on the first drug's real-time drug concentration data, future pharmacokinetic parameters associated with the first drug in the patient. In embodiments where the catheter-based electrochemical sensor is also used to detect the second drug, hardware processor 304 may execute instruction 310 to also predict future pharmacokinetic parameters associated with the second drug in the patient. As used herein, pharmacokinetic parameters may refer to parameters related to the behavior of a drug in the patients body (e.g., drug concentration vs. time).

As described above, in various embodiments hardware processor 304 may use Bayesian statistics (or Bayesian statistical models) to make these predictions. Bayesian statistical methods use Bayes' theorem to compute and update probabilities after obtaining new data. Because Bayesian statistical methods/models are designed to update probabilities after obtaining new data, they are well-suited for making pharmacokinetic parameters predictions based on the real-time drug concentration data obtained from catheter-based electrochemical sensor. In other words, Bayesian methods/models may constantly adapt and improve their predictions in response to new patient-specific drug concentration data obtained from the catheter-based electrochemical sensor.

Accurate predictions about future pharmacokinetic parameters for a patient (e.g., future in-vivo concentrations of an anesthetic drug) can be invaluable in clinical settings such as surgical operations. When administering drugs, clinicians must consider the past (e.g., what happened after a dose was administered), present (e.g., current status of the patient), and future (e.g., when does the patient need to be awakened from anesthesia, how much longer is adequate pain control required, etc.). While it can be extremely helpful for a clinician to have accurate drug concentration data for the past and present, in order to administer drugs appropriately the clinician must also consider future time points. In other words, to ensure that future drug concentrations are maintained within target ranges, a clinician (or an automated system such as hardware processor 304) must make appropriate interventions in the present. By providing accurate predictions for a patient's future pharmacokinetic parameters, embodiments can inform and improve these interventions immeasurably.

In various embodiments, hardware processor 304 may utilize additional patient-specific data to predict future pharmacokinetic parameters. In addition to the real-time drug concentration data obtained by the catheter-based electrochemical sensor, this data may comprise demographic information of the patient (e.g., height, weight, age, gender) pre-operative physiological or laboratory data of the patient (e.g., renal function data, liver function data, blood pressure, glucose data, hemoglobin data, etc.), and monitored/dynamic physiological data of the patient (e.g., monitored/dynamic measurements of renal function data, liver function data, processed or raw EEG data, blood pressure, cardiac output, glucose data, hemoglobin data, etc.). In various embodiments, hardware processor 304 may learn how to weight these various input variables when predicting future pharmacokinetic parameters. In other words, hardware processor 304 may learn how these input variables influence future pharmacokinetic parameters and adjust pharmacokinetic model weights/parameters in accordance with this learning.

Hardware processor 304 may execute instruction 312 to adjust, based on the first drug's predicted pharmacokinetic parameters, administration of the first drug to the patient. In embodiments where the catheter-based electrochemical sensor also predicts pharmacokinetic parameters for the second drug, hardware processor 304 may execute instruction 312 to adjust administration of the second drug to the patient. Adjusting administration of the first/second drug may comprise increasing, decreasing or stopping infusion for the drug, bolusing the drug, etc.

In various embodiments, instead of, or in addition to, adjusting administration of a drug, hardware processor 304 may provide a medical-related notification to a clinician based on its predictions of future pharmacokinetic parameters.

This medical-related notification may take various forms and contain various types of medical-related information. For example, the medical-related notification may comprise an alert/recommendation to verify that infusion is appropriately connected to patient, increase or decrease infusion rate for a drug, bolus a drug, stop infusion to prepare to wake the patient up in a certain amount of time, etc. In other examples the medical-related notification may display various types of predictive information such as curves representing future concentrations of a drug vs. time (i.e. pharmacokinetic curves), or curves representing future effects of the drug vs. time (i.e. pharmacodynamic curves), etc. The display may include the range and likelihoods of future concentrations or effects. The display may show the predicted effect and likelihood of suggested interventions such as bolusing a medication, increasing or decreasing or stopping an infusion, etc.

In certain jurisdictions, embodiments may provide medical-related notifications to a clinician instead of adjusting administration of drugs automatically in order to ensure patient safety. For example, in the U.S., FDA regulations related to patient safety require a physician to be the ultimate decision maker when administering drugs to a patient. Behind these regulations is a belief that a clinician may be able to take into account contextual factors such as what is happening with the procedure, hemodynamic responses to prior interventions, and the entire range of possible concentrations at any given point in time better than computer/automated system would. Appreciating this clinical reality, embodiments of the presently disclosed technology may provide a medical-related notification to a clinician instead of adjusting administration of a drug automatically.

The following sections of the present disclosure describe examples of the electrochemical sensor of the present disclosure in greater detail. These sections also describe example experiments conducting in accordance with embodiments of the presently disclosed technology.

In order to realize a continuous monitoring system to enable simultaneous in-vivo measurements of both drugs directly in human blood, several challenges should be addressed. These include the vastly different micromolar (μM) and nanomolar (nM) concentration ranges of the target PPF and FTN, respectively, related cross talk, a high degree of fouling during PPF oxidation due to the electrode passivation by the polymerized reaction products, and the extremely low detection limits associated with the nM FTN concentration, along with the substantial biofouling expected in the complex blood matrix.

Some embodiments demonstrate the first example of a microcatheter-based dual-analyte sensor capable of continuous simultaneous electro-chemical monitoring of PPF and FTN in connection to square-wave voltammetric detection. In some embodiments, a microcatheter-based simultaneous sensing platform can rely on embedding two Teflon-based microtubes, packed with different modified CP materials as working electrodes combined with the Ag/AgCl reference electrode wires inside an external Teflon tube. In some embodiments, the CP transducer materials are modified to tune the sensitivity and dynamic range of each drug and address challenges for realizing such simultaneous monitoring of these anesthetic agents. In some embodiments, such electrode modification involved the use of a PVC-coated CP electrode for PPF and a Carbon Nano Tube (CNT)-incorporated Carbon Paste (CP) transducer coated with multilayers of Au nanoparticles/erGO hybrid and PVC outer layers for FTN. In some embodiments, the resulting dual microcatheter sensor exhibits an attractive analytical performance in protein-rich artificial plasma and in un-treated human blood samples, with excellent selectivity against interferents, and sensitive and stable response suitable for continuous monitoring. Examples of performance characteristics of the PPF/FTN microcatheter sensor are discussed in the following sections, along with the prospects and challenges for practical safe administration of these drugs during anesthesia in operating rooms.

Chemicals: Propofol (PPF), fentanyl citrate salt solution (100 μg/mL, supplied in methanol, FTN), ascorbic acid (AA), acetaminophen (AP), caffeine (CFN), glucose (GL), uric acid (UA), theophylline (TPL), multi-walled carbon nanotubes (CNT, ≥90% carbon basis), polyvinyl chloride (high molecular weight, PVC), gold (III) chloride trihydrate (HAuCl4.3H2O), chloride (FeCl3), bovine serum albumin (BSA), γ-globulins from bovine blood, phosphate buffer solution (PBS) (1.0 M, pH 7.4), calcium chloride anhydrate (CaCl2)), potassium chloride (KCl), magnesium sulfate anhydrous (MgSO4), mineral oil, sodium sulfate anhydrous (Na2SO4), sodium bicarbonate (NaHCO₃), sodium chloride (NaCl), sodium hydroxide (NaOH), sodium phosphate monobasic (NaH2PO4), sodium phosphate dibasic (Na2HPO4), sodium nitrate (NaNO3), sodiumL-lactate (Lac), hydrochloric acid (HCl) and sulfuric acid (H2SO4) may be used. Graphite powder (crystalline 99%) may be used. Graphene oxide (GO) may be used. Tetrahydrofuran (THF) and methanol may be used. Artificial plasma may be prepared by dissolving certain amounts of different electrolytes, including NaCl, CaCl2), KCl, MgSO4, NaHCO₃, Na2HPO4, and NaH2PO4. In some embodiments, the BSA and γ-globulin proteins were also added (at 2 mg/mL each), to mimic the proteinaceous texture of the blood. Human blood samples, in some examples, may be kept at 4° C. prior to use. In some embodiments, untreated blood samples were used in the PPF and FTN detection experiments. In some embodiments, the PPF solutions for performing electroanalysis may be diluted in PBS or artificial plasma prior to use from a stock solution of 10 mM PPF prepared in 0.1 M NaOH. In some embodiments, FTN solutions were diluted in PBS or artificial plasma from the original stock solution.

In some embodiments, electrochemical measurements were carried out at room temperature in a 2 mL volume homemade cell containing the analysis medium (PBS (0.1 M, pH 7.4), or artificial plasma (pH 7.4) or untreated blood samples by using a hand-held potentiostat (PSTrace software version 5.6). In some embodiments, the integrated dual-sensor micro-catheter, including WP (WE for propofol), WF (WE for fentanyl), RP (RE for propofol) and RF (RE for fentanyl) (FIG. 1A), were operated in a two-electrode system using square wave voltammetric (SWV) technique with optimized parameters of 10 Hz frequency, 50 mV amplitude, 4 mV step potential and 1 min accumulation time. Background subtraction SWV FTN detection experiments were performed with PSTrace software version 5.6.

Fabrication of microcatheter sensors: In one example, Wp may be prepared by packing the tip of a 5-cm-long Teflon tube (0.3 mm inner diameter, 0.6 mm outer diameter) with carbon paste (65% carbon paste and 35% mineral oil, w/w) up to a final height of 3 mm. In some embodiments, the surface may be smoothed by gently rubbing the tip to a fine piece of paper. After that, in some embodiments, its inner end may be connected with a 7-cm-long copper wire (0.2 mm diameter) for electrical contact. In some embodiments, a 0.2 μL of PVC solution (20 mg PVC in 5 mL THF) may be drop casted and kept at room temperature for further experiments.

In some embodiments, WF may be obtained by first incorporating 2% CNT in graphite and then, making the CP transducer by mixing the as-prepared solid material with mineral oil (65%:35%, w/w), followed by packing a Teflon tube with the conductive paste and externally connecting in the same way as used for Wp. The surface may be drop casted by 0.2 μL of PVC solution. The obtained electrode may be referred to as PVC/CNT-CP. In some embodiments, for ultrasensitive FTN detection, a multilayered modification protocol may be designed. In one example, Au nanoparticles is electrochemically deposited on the CNT-CP surface in 0.1 M NaNO3 solution containing 3 mM HAuCl4 by applying chronoamperometry at +0.2 V for 2 min (Au/CNT-CP). After gentle rinsing with DI water, in one embodiment, the platform surface may be modified with the electrochemically reduced graphene oxide (erGO) nanosheets by using cyclic voltammetry (CV) involving potential scanning over the 0.3 to −1.5 V range for 5 cycles in 0.1 mg/mL GO solution (0.1 M H2SO4 and 0.5 M Na2SO4) [19] and referred to as erGO/Au/CNT-CP. In some embodiments, 0.2 μL of PVC solution may be drop casted to give the final multilayered design, referred to as PVC/erGO/Au/CNT-CP.

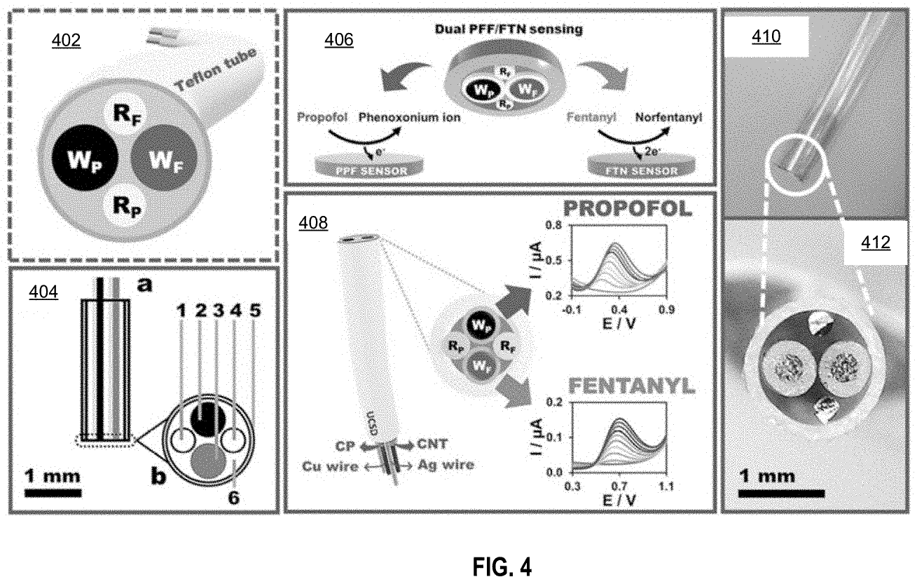

In some embodiments, for simultaneous PPF/FTN measurements, the integrated dual-sensor catheter, including the working electrodes for both analytes along with the corresponding reference electrodes (FIG. 4A), may be immersed in the relevant sample matrix, followed by successively spiking mixture solutions containing different concentrations of the two drugs. SWV measurements may be performed by recording the voltammetric response of PPF first, followed by immediate recording the FTN signal.

In some embodiments, the design of the integrated dual microcatheter sensor is schematically presented in FIG. 4. Such scheme illustrates the integrated microcatheter sensor (FIG. 4, block 402) along with its cross-sectional view (FIG. 4, block 404) and shows the two WEs (WP, WF) and two REs (RP, RF), tightly inserted into a 5-cm-long Teflon tube (1.3 mm inner diameter, 1.6 mm outer diameter). In some embodiments, the 7-cm-long Ag/AgCl wire REs is obtained by cutting an Ag wire and chloridation through reacting with 0.1 M FeCl3 for 1 min. FIG. 4, block 406, depicts the principle of simultaneous SWV monitoring of PPF/FTN on the integrated dual microcatheter sensor, with the corresponding oxidation reactions at the WEs, while FIG. 4, block 408, shows representative SWV curves obtained in the presence of increasing levels of the target anesthetic drugs over the 0-90 μM range. The optical images of the dual microcatheter sensor were shown in FIG. 4, block 410, 412.

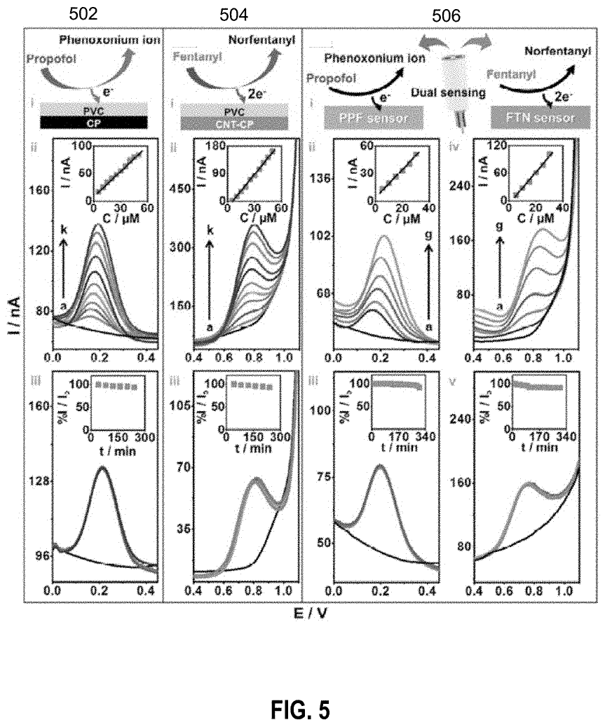

Individual PPF detection: In some embodiments, the analytical performance of the developed microcatheter sensor may be first evaluated individually for each anesthetic agent. The resulting optimum operating conditions may be subsequently used toward the simultaneous dual-analyte measurements. The PPF detection on the catheter sensor may rely on monitoring its oxidative reaction, as shown in FIG. 5, block 502. Here, the phenolic group in the PPF may undergo a single-electron oxidation to give the phenoxonium ions. These ions can initiate other unwanted reactions which result in electrode passivation due to the formation of polymerized films on the surface and thus, fouling of the electrode. Such fouling effect may limit the establishment of a sensory device for long-term continuous measurements. In some embodiments, intermittent cleaning strategies carried out after every six measurements to remove the fouling-associated problems on boron-doped diamond or pencil graphite electrodes. However, these cleaning strategies, based on running CVs in an alkaline solution or high-potential amperometric treatments in PBS, may not be adaptable for in-vivo measurements and may also offer poorer reproducibility. Another effort toward avoiding electrode fouling during PPF analysis may be achieved by using a plasticized organic film including a cocktail mixture of PVC, ion exchanger, organic electrolyte, and plasticizer. The success of this approach may be based on high partitioning of the lipophilic PPF inside the lipophile organic layer as well as dissolving ability of the film for possible fouling-inducing molecules and ions. In some embodiments, the use of such a complex mixture with many ingredients may affect the fabrication reproducibility of the sensor. The high fouling-resistant properties of PVC has long been established by in connection to oxidase enzyme-based sensors for monitoring NAD-dependent dehydrogenase enzymatic reactions. The results of the presently disclosed technology, demonstrate the characteristics of the PVC outer layer toward selective and sensitive PPF detection along with minimal fouling effects.

In some embodiments, SWVs were recorded at the PVC/CP catheter sensor in artificial plasma solution containing increasing PPF concentrations over the range of 5-50 μM. As shown in FIG. 5, block 502(ii), the PPF oxidation peak current, appeared at around +0.2 V, increased linearly upon raising the PPF concentrations over the entire concentration range. As the usual time to complete a surgical operation is around 4 h, the long-term stability of the sensor may be evaluated over such period in the presence of 20 μM PPF, by recording intermittent SWVs at 40 min intervals. The results shown in FIG. 5, block 202(iii) illustrate that the sensor retains 95% of its original current signal after 4 h continuous operation, indicating an extremely high sensor stability toward PPF analysis and reflecting the effective antifouling properties of the protective PVC electrode coating.

Individual FTN detection: The fabrication of electrochemical FTN monitoring platforms recently gained considerable attention, which may be due to the number of death tolls resulting from overdose of this potent drug that necessitates easy-to-use monitoring systems to facilitate a rapid life-saving intervention by clinical personnel. Here, one example may include a catheter-based sensor based on a CNT-incorporated CP-packed transducer toward FTN detection in artificial plasma samples. As shown in FIG. 5, block 504(i), the FTN detection relies on its oxidation reaction on the electrode surface, giving rise to a unique single peak at around +0.7 V. During this reaction, FTN oxidizes to norfentanyl through a 2e−—2H+ oxidation mechanism, including oxidative dealkylation of the piperidine tertiary amine [25]. FIG. 5, block 504(ii) shows the SWVs recorded at the PVC/CNT-CP catheter electrode in an artificial plasma medium containing increasing levels of FTN. An evident growth in the oxidation currents were observed due to the successive FTN spiking into the solution. A linear calibration plot may be obtained for FTN over the entire studied range from 5 to 50 μM. A fouling-resistant coating strategy by using a PVC organic layer may be developed to achieve long-term stability during electrochemical FTN monitoring experiments FIG. 5, block 504(iii). Such high stability may be demonstrated using intermittently recorded SWV responses for 20 μM FTN in artificial plasma fluid during a prolonged 4 h of sensor operation. The results of FIG. 5, block 502(iii) indicate the FTN catheter sensor may be able to retain 94% of its original current response after such prolonged continuous operation in artificial plasma.

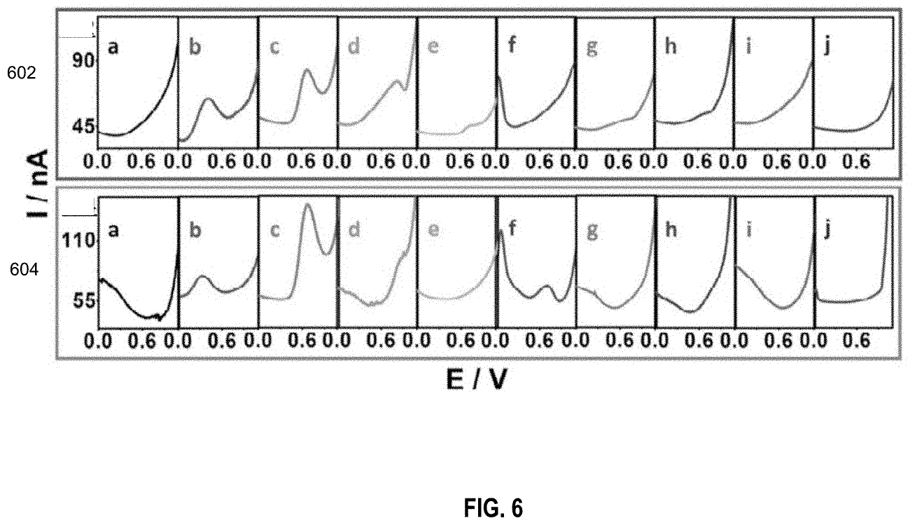

Simultaneous dual-analyte PPF/FTN analysis: In some embodiment, optimizing the performance of sensors individually, the integrated dual catheter-based sensor may be assessed to assure the feasibility of multiplexed measurements without any cross-reactivity between two analytes. FIG. 5, block 506(i) illustrates the integrated dual catheter sensor and the relevant reaction mechanisms occurring on the neighboring electrodes during such simultaneous PPF/FTN detection. The response characteristics of the integrated catheter sensor toward concurrently increasing concentrations of PPF/FTN over the range of 5-30 μM may be recorded. The corresponding SWVs, displayed in FIG. 5, block 506(ii), confirm minimal cross-reactivity between the two drugs and thus, the potential of the platform toward such simultaneous measurements of these anesthetic agents. In addition, the long-term operation stability of the integrated dual sensor may be evaluated by spiking 20 μM of each drug in the presence of the same level of the second one and recording SWVs during 5 h of operation in 15 min intervals (FIG. 5, block 506(iii)). Experiments show more than 80% of the current response retained during detecting of both drugs after 5 h. Additionally, the selectivity of the catheter-based sensors may be examined against many potential endogenous and exogenous interfering species, including ascorbic acid, lactate, acetaminophen, uric acid, caffeine, glucose and theophylline. FIG. 6 block 602, 604 present the SWV data obtained at PVC/CP electrode (FIG. 6 block 602) and PVC/CNT-CP electrode (FIG. 6 block 604) upon spiking of 20 μM of the target analytes along with 150 μM excess of interferent compounds. The result indicates that almost all studied species do not interfere during the analysis of the PPF/FTN. The high selectivity of the catheter sensor toward detecting PPF and FTN may be attributed to the organic PVC film which allows these drugs to be partitioned through this coating and undergo the redox reactions, whereas the film repels interfering species with lower lipophilicity. Note, however, the small oxidation peaks observed for the large excess of ascorbic acid (FIG. 6 block 602,d) and acetaminophen (FIG. 6, block 604,f) on the PVC/CP and PVC/CNT-CP electrodes, respectively. Such small interferences may be addressed by adjusting the PVC polymeric layer density.

PPF/FTN analysis at the optimal target ranges: One embodiment reported screen-printed carbon electrodes and glove-based flexible wearable sensors, based on the use of room temperature ionic liquids (RTIL), toward fast, on-the-spot field detection of μM concentrations of FTN. An improvement in the FTN sensing characteristics may be realized by using micro-needle-based electrodes modified by a layered nanomaterials-based protocol for nM-range, in-vivo FTN monitoring applications. Further improvements of such a unique system are shown through its integration with PPF sensor in a microcatheter-based strategy toward both sensitive and stable simultaneous, continuous monitoring of these anesthetic drugs.

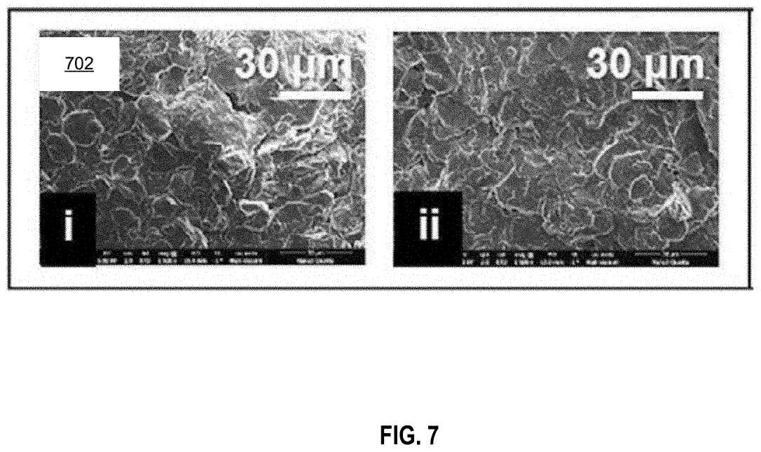

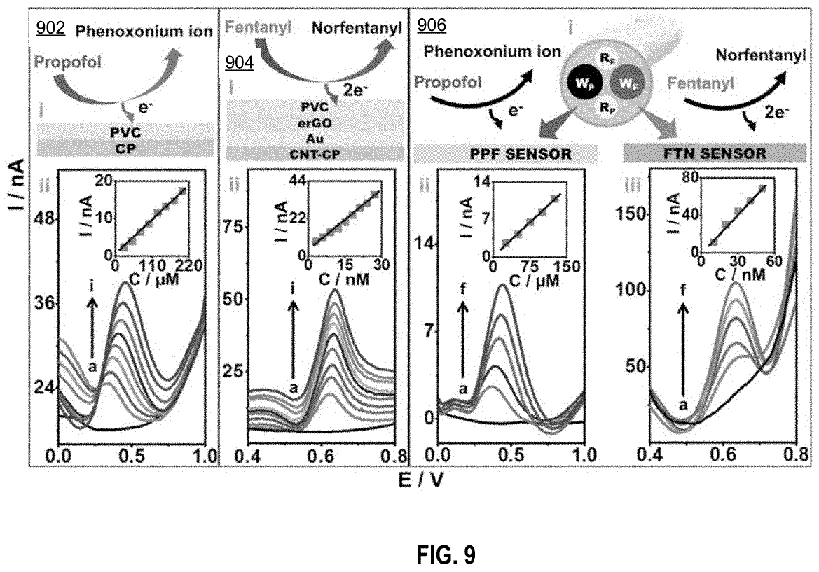

While the different oxidation potentials of PPF and FTN make it possible to detect both drugs on the same working electrode, the different (μM and nM) concentration ranges of these target analytes requires fine tuning the composition of the individual working electrodes for meeting the corresponding sensitivity requirements, and hence to rely on a miniaturized dual catheter platform towards such simultaneous detection. A portable microcatheter sensor may be used for directing in-vivo monitoring of PPF and FTN in human plasma should be able to cover different concentration ranges of the target drugs, that is ˜25-175 μM for PPF and ˜1-40 nM for FTN. One example shows that by using simple modification protocols, the performance of the sensor can be tailored to detect the target plasma levels of these analytes. PPF detection may be achieved through a PVC-modified CP electrode catheter. FIG. 7, block 702(i,ii) show the SEM images of CP and PVC-modified CP electrodes. A uniform structural morphology may be seen in these images. In order to broaden the dynamic range for PPF, the concentration of the outer PVC membrane may be optimized. It may be found that a denser PVC layer (40 mg dissolved in 5 mL THF) produces a linear concentration dependence for PPF levels up to 200 μM. FIG. 9, block 902, shows the SWVs obtained at the CP catheter sensor coated with such a dense PVC layer upon addition of PPF in the concentration range of 25-200 μM in 25 μM increments. The linear calibration plot demonstrates the potential of the catheter sensor to detect PPF over the entire range of interest. The limit of detection (LOD) may be estimated as 4.3 μM (S/N=3).

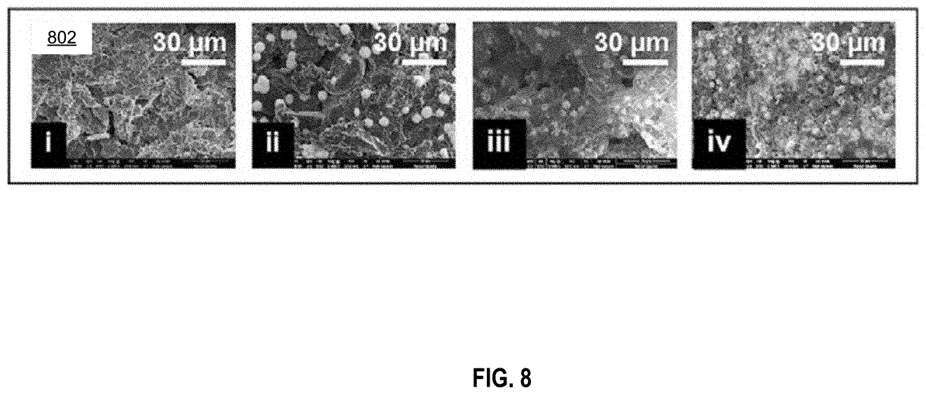

In some embodiments, to enhance the sensitivity of FTN detection system toward ultra-sensitive (nanomolar) sensing, the high catalytic efficiency of Au nanoparticles may be combined with the attractive electron conductivities of carbon-based nanomaterials, including graphene sheets and carbon nanotubes. FIG. 8, block 802(i) presents the SEM image of the underlying electrode including 2% CNT-incorporated CP. A uniform distribution of Au nanoparticles may be deposited on the electrode surface through electrochemically reducing Au3+ cations (FIG. 8, block 802 (ii)). This may be followed by deposition of graphene nanosheets through electrochemical reduction of a GO suspension using potential-scanning CV technique. FIG. 8, block 802 (iii) shows the formation of netlike structure of such graphene sheets on the surface. The high surface area offered by the graphene-Au hybrid can create more surface-active sites towards the FTN redox reaction. Graphene nanosheets not only causes the FTN molecules to pre-concentrate on the electrode surface through hydrophobic n-n interactions, but also stabilize the Au nanoparticles and creates a network of fast electron conduction pathways between the catalytic Au nanoparticles. Similar to the PPF sensor, a PVC outer layer may be employed for the FTN detection to impart the selectivity and anti-fouling properties to the sensor (FIG. 8, block 802(iv)). FIG. 9, block 904(i) schematically illustrates the multi-layered surface structure of the FTN catheter sensor towards ultra-sensitive (nM) detection. As illustrated in FIG. 9 block, 904(ii), the resulting sensor offers a well-defined SWV response to increasing additions of 3 nM FTN. The corresponding calibration curve, shown as inset, displays a linear response behavior of FTN sensor over the 3-24 nM concentration range, indicating a tremendous promise for ultrasensitive FTN detection. LOD for FTN catheter sensor may be calculated as 2.18 nM (S/N=3).

The simultaneous dual analyte PPF/FTN detection may also be investigated using the integrated dual catheter sensor prepared with the modification protocols, shown in FIG. 9, block 902, 904. A mixed solution containing relevant concentrations of both FTN (1 μM) and PPF (2.5 mM) analytes may be prepared accordingly and spiked in 1:100 dilutions into the solution. The detection of PPF may be examined over the 25-125 μM range while the FTN may be assessed between 10 and 50 nM. Similar to individual detection schemes, a linear correlation between voltammetric current response and the concentration of analytes may be observed over the entire studied range. Interestingly, for both analytes, the current signals produced for a given concentration under individual and simultaneous detection modes were quite similar, confirming further the negligible cross-reactivity between the detection procedures of PPF and FTN. The herein obtained data demonstrates that the dual catheter sensor may be suited to the simultaneous detection of dual PPF/FTN analytes at their relevant concentrations.

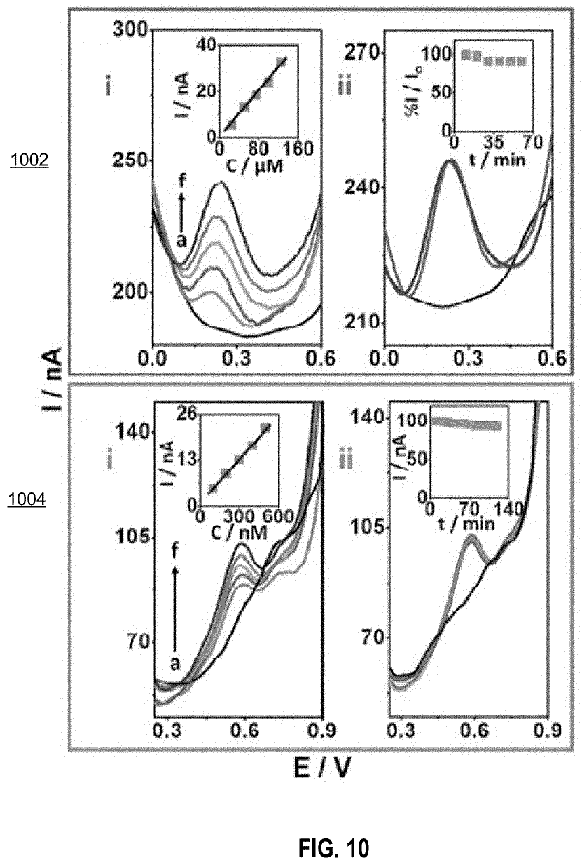

Individual PPF/FTN detection in whole blood medium: Towards the ultimate goal of applying the integrated microcatheter sensor toward direct multiplexed in-vivo monitoring of anesthetic drugs, some embodiments evaluate the performance of the dual microcatheter sensor in whole blood samples. For example, FIG. 10, block 1002(i) displays SWVs obtained upon spiking PPF into the blood sample in 25 μM increments over the range of 25-125 μM, along with the corresponding calibration plot (inset). These data indicate that the sensor responds favorably and linearly to the PPF additions over the entire range. The stability of such whole blood PPF measurements may be examined by intermittently recording SWV response of the sensor toward 100 μM PPF at 10 min intervals. The results, shown in FIG. 10, block 1002(ii), illustrate that the sensor can retain >80% of the original current response after 1 h, after which a larger decrease of the response may be noticed. Similarly, the sensitivity and operational stability of the PVC/erGO/Au/CNT-CPE sensor were tested towards direct detection of nanomolar FTN concentrations in the whole blood samples. The result, presented in FIG. 10, block 1004, demonstrate the satisfactory sensing performance along with stable current response during 2 h continuous operation of the sensor.

Conclusions: Multiplexed detection of clinically-important analytes recently attracted considerable interest as it offers more comprehensive information about a specific disease compared to the single-analyte measurements. Embodiments include the multiplexed micro-needle detection of ketone bodies along with glucose and lactate, or a dual glucose/insulin microchip platform toward advanced diabetes management. Despite the urgent need for an analytical platform toward simultaneous real-time measurement of the widely used PPF and FTN drugs during surgical operations toward a timely and efficient personalized dose optimization, such dual-analyte sensing are not reported. Compared to early multiplexed sensors, such surgical operations use real-time blood monitoring. To address this challenge, the present embodiments demonstrate an integrated microcatheter-based dual sensing probe towards continuous in-vivo or in a sample removed from the patient monitoring of plasma concentrations of propofol and fentanyl. In some embodiments, the microcatheter sensor, may rely on electrochemical two-electrode system with SWV transduction method, exhibit an analytical performance with sensitive linear response within the desired μM and nM concentration ranges for PPF and FTN, respectively, along with high selectivity, stability and speed in both protein-rich artificial plasma and in untreated blood samples. The results indicate the benefits of such a device towards continuous drug monitoring during surgeries and a real-time safety alert for patients receiving these drugs for anesthesia and procedural sedation. It should be appreciated that the surface coating may be further improved to impart higher selectivity and protection against biofouling by the integration of a miniaturized dual potentiostat for simultaneous real-time PPF and FTN measurements, and a large-scale validation of the microcatheter sensing platform against gold-standard GC-MS or LC-MS centralized methods. In some embodiments, the dual-sensor catheter may be incorporated into a closed-loop feedback-controlled anesthesia system towards a timely responsive personalized administration of PPF and FTN during surgical procedures. The application scope of the microcatheter sensor can also be expanded to include additional anesthetic drugs for further medical safety control and thus, towards enhanced patient comfort.

In some embodiments, the disclosed technology can be used with AI or reinforcement learning algorithms to guide infusion rates or other features. While implementations and examples are described, it should be appreciated that other implementations, enhancements, and variations can be made based on what is described and illustrated in this patent document.

The following sections include further descriptions of certain example figures.

FIG. 4. The schematic illustration of dual PPF/FTN sensing on integrated microcatheter sensor. (Block 402) Integration of dual microcatheter sensor for PPF/FTN detection. (Block 404) Cross-sectional view of the microcatheter sensor tip: 1, Ag/AgCl; 2, PPF sensor; 3, FTN sensor; 4, Ag/AgCl; 5, Teflon tubing wall; 6, Interior of Teflon tube. (Block 406) Schematic illustration of simultaneous PPF/FTN sensing on the integrated microcatheter sensor. (Block 408) The schematic representation of side-view and top-view combinations; two Ag/AgCl wires as reference electrodes for PPF (R_{P}) and for FTN(R_{E}), a CP microcatheter electrode as the working electrode for PPF (W_{P}) and the CNT-CP microcatheter electrode as the working electrode for FTN (W_{F}), along with the recorded SWV PPF/FTN monitoring from 0 to 90 μM in 0.1 M PBS pH 7.4 vs. integrated Ag/AgCl wires. (Block 410) The whole-body photo image of the integrated dual microcatheter sensor along with (Block 412) the cross-section image of the integrated dual microcatheter sensor.

FIG. 5. Detection of PPF/FTN at integrated dual microcatheter sensor in protein-rich artificial plasma medium. (Block 502) PPF analysis at PVC/CP electrode microcatheter sensor. (i) Schematic illustration of PPF detection on the microcatheter sensor. (ii) SWVs recorded in artificial plasma (a) upon spiking with 5 μM increments of PPF (5-50 μM) (b-k). (iii) Stability investigation of the microcatheter sensor in 20 μM PPF by performing repetitive measurements at 40 min intervals in artificial plasma over a period of 4 h. (Block 504) FTN analysis on PVC/CNT-CP electrode microcatheter sensor. (i) Schematic illustration of FTN detection on the microcatheter sensor. (ii) SWVs in artificial plasma (a) and in different concentrations of FTN added to the solution in 5 μM increments (5-50 μM (b-k)). (iii) The stability performance of the sensor in 20 μM FTN; six repetitive measurements were recorded at 40 min intervals over a period of 4 h. (Block 506) Simultaneous PPF/FTN mixture analysis on integrated PVC/CP and PVC/CNT-CP microcatheter sensor. (i) Schematic illustration of simultaneous PPF/FTN analysis on the integrated microcatheter sensor. (ii) Sequentially recorded SWVs of PPF (left) and FTN (right) sensors in artificial plasma solution (a) and after addition of different mixed concentrations of PPF/FTN (b-g) (5-30 μM in 5 μM increments). (iii) Stability performance of the PPF sensor in mixture solution of PPF and FTN, 20 μM each; twenty repetitive measurements were recorded at 15 min intervals over a period of 5 h. (iv) Stability performance of FTN sensor in a mixed PPF/FTN solution, 20 μM each; twenty repetitive measurements were recorded at 15 min intervals over a period of 5 h.

FIG. 6. Selectivity investigation of the (Block 602) PVC/CP and (Block 604) PVC/CNT-CP sensors against various potential interferents. SWVs were recorded in (a) artificial plasma upon adding (b) 20 μM PPF, (c) 20 μM FTN and 150 μM of each interfering species, including (d) ascorbic acid, (e) lactate, (f) acetaminophen, (g) uric acid, (h) caffeine, (i) glucose and (j) theophylline.

FIG. 7. The detection of PPF/FTN at the optimal target levels realized through adjustments in modification protocols. (block 702) SEM images of (i) bare CP catheter electrode surface and (ii) PVC/CP electrode used for PPF detection.

FIG. 8. (block 802) The detection of PPF/FTN at the optimal target levels realized through adjustments in modification protocols. SEM characterization of the fabrication steps of the FTN catheter sensor; (i) CNT-CP, (ii) Au/CNT-CP, (iii) erGO/Au/CNT-CP and (iv) PVC/erGO/Au/CNT-CP.

FIG. 9. (block 902) PPF analysis on PVC/CP microcatheter sensor. (i) Schematic illustration of PPF monitoring on the microcatheter sensor. (ii) The SWVs recorded in protein-rich artificial plasma solution (a) and upon addition of 25 μM increments of PPF (25-200 μM) (b-i). (block 904) FTN analysis on PVC/erGO/Au/CNT-CP microcatheter sensor. (i) Schematic illustration of FTN monitoring on the microcatheter sensor. (ii) SWVs recorded in artificial plasma solution before (a) and after adding 3 nM increments of FTN (3-24 nM) (b-i). (block 906) Simultaneous PPF/FTN mixture analysis on integrated PVC/CP and PVC/erGO/Au/CNT-CP microcatheter sensor. (i) Schematic illustration of simultaneous PPF/FTN monitoring on the integrated microcatheter sensor. (ii) SWVs obtained at the PPF sensor upon addition of a mixed solution of PPF/FTN (2.5 mM PPF/1 μM FTN) in the range of 25-125 μM in 25 μM increments (a to f). (iii) SWVs of FTN sensor recorded in artificial plasma solution while adding mixed concentrations of PPF and FTN in the range of 10-50 nM. SWV potential ranges; 0-1 V for PPF and 0.4-0.8 V for FTN.

FIG. 10. Individual PPF/FTN detection in whole blood medium. (block 1002) Individual PPF analysis on PVC/CP microcatheter sensor. (i) SWVs of PPF (25-125 μM, 25 μM increments) (a to f) with relative peak current vs. concentration recorded in whole blood sample (inset). (ii) Stability performance of 100 μM PPF; six repetitive measurements were recorded at 10 min intervals over 1 h period; Change in relative peak current percentage vs. time (inset). SWV potential range, 0-0.6 V vs. integrated Ag/AgCl wires. (block 1004) Individual FTN analysis on PVC/erGO/Au/CNT-CP microcatheter sensor. (i) SWVs of FTN (100-500 nM, 100 nM increments) (a to f); relative current response vs. concentration recorded in whole blood sample (inset). (ii) Stability performance of 500 nM FTN; twelve repetitive measurements were recorded at 10 min intervals over a period of 2 h; Change in relative percentage of current response vs. time. SWV potential range: 0.25-0.9 V vs. integrated Ag/AgCl wires.

Additional information on example methods, systems, and devices in accordance with the present technology are described below.

According to the American Society of Anesthesiologists Closed Claims Database, one of three drug-related errors is the result administrating an incorrect dose. Directly measuring drug concentration removes the uncertainty in the dose-concentration relationship and addresses inter- and intra-subject variabilities that affect the pharmacokinetics of anesthetics. In the presently disclosed technology, some embodiments describe a dual-analyte microcatheter-based electrochemical sensor capable of simultaneous real-time continuous monitoring of fentanyl (FTN) and propofol (PPF) drugs simultaneously in the operating rooms. Such a dual PPF/FTN catheter sensor may rely on embedding two different modified carbon paste (CP)-packed working electrodes along with Ag/AgCl microwire reference electrodes within a mm-wide Teflon tube and use a square wave voltammetric (SWV) technique. The composition of each working electrode is designed to cover the concentration range of interest for each analyte. A polyvinyl chloride (PVC) organic polymer coating on the surface of CP electrode enabled selective and sensitive PPF measurements in μM range. The detection of nM FTN levels is achieved through a multilayered nanostructure-based surface modification protocol, including a CNT-incorporated CP transducer modified by a hybrid of electrodeposited Au nanoparticles and electrochemically reduced graphene oxide (erGO) and a PVC outer membrane. The long-term monitoring capability of the dual sensor may be demonstrated in a protein-rich artificial plasma medium. The promising antibiofouling behavior of the catheter-based multiplexed sensor may also be illustrated in whole blood samples. The integrated dual-sensor microcatheter platform can be used in realtime, in-vivo detection of the anesthetic drugs, propofol and fentanyl, during surgical procedures towards improved safe delivery of anesthetic drugs.

Example Aims: Some embodiments will incorporate the direct drug measurements from the catheter into a closed-loop drug delivery system, capable of using these direct concentration measurements as feedback parameters. In some embodiments, the purpose of closed-loop anesthesia may be to link observation with intervention, with the theoretical benefit of finer and more accurate control. Closed-loop drug delivery may be demonstrated to have improved performance over open-loop control. Closed-loop delivery of propofol and the opioids remifentanil and alfentanil have been studied. These closed-loop models utilized depth of anesthesia monitors or hemodynamic variables including heart rate and blood pressure as input variables of the loop. Closed-loop drug delivery may be dependent on a reliable feedback from a sensor to adjust the rate of drug delivery. To date, the most commonly used feedback control systems is depth of anesthesia monitors and patient hemodynamic parameters. Hemodynamic parameters may be subject to vast amounts of variability secondary to surgical, anesthetic, and physiologic perturbations associated with a surgical procedure. Depth of anesthesia monitors may be limited in their ability to guide titration of anesthesia in the clinical setting. These monitors may be subject to confounding secondary to electromyographic and pharmacologic interference as well as hysteresis.

This catheter can continuously and simultaneously measure in real-time, in-vivo concentrations of propofol and fentanyl. To date, the anesthetic agents whose concentrations can be measured continuously and in real-time are volatile anesthetic agents. There exists no such technology that allows measurement of intravenously administered drugs. Administration of intravenous hypnotics and opioids will no longer be performed in the “dose domain”. While existing target controlled infusions utilize mathematical models that (theoretically) allow administration of drugs within the “concentration domain”, the presently disclosed technology will capitalize on such models but use real-time measurements to improve accuracy. Furthermore, since all aspects of the pharmacokinetic (concentration-time) and pharmacodynamic (concentration-effect) relationships will be measurable, drug concentration (not dose) will be targeted and can be correlated to each subject's observed effect, truly ushering personalized medicine.

Moreover, this device can monitor two drugs at once via a double sensing platform of a single integrated dual microcatheter sensor. This sensing platform can offer electrochemical information on the two target drugs by using rapid and sensitive square wave voltammetry (SWV) at the optimized conditions. In certain embodiments, the designed platform of this sensor may be constructed from the combination of two different internal Teflon tubes containing judiciously modified carbon electrodes as working electrodes for each target analyte along with the corresponding Ag wires as reference electrodes. These electrodes may be inserted with an external Teflon tube as an integrated dual microcatheter sensor. In some embodiments, the novel electrode surface coatings (using various polymeric and nanomaterials) can impart high selectivity and sensitivity of both analytes, while preventing bio-fouling in and extending the stability whole blood.

In addition, in some embodiments a closed-loop drug delivery system for propofol and fentanyl may incorporate the measurements provided by the catheter. This embodiment may replace target controlled infusions, which rely on mathematical models alone to predict plasma and/or effect-site concentrations as pharmacokinetic endpoints. In some embodiments, the real-time, in vivo concentration measurements may be true pharmacokinetic inputs into the closed loop system. Prior closed-loop systems of anesthetic rely solely on effect endpoints (i.e. pharmacodynamic endpoints) as a feedback. These surrogate markers of effect, including depth of anesthesia monitors and hemodynamic variables may be subject to confounding and are insufficient for sole use as a feedback control. Certain embodiments may incorporate measured drug concentration as a feedback control mechanism in a closed-loop system.

Certain embodiments may provide a real-time, measured relationship between drug concentration versus time (pharmacokinetics) and drug concentration versus effect (pharmacodynamics). By doing so, individual PK-PD models for each patient will be constructed and incorporated into their respective electronic medical record.

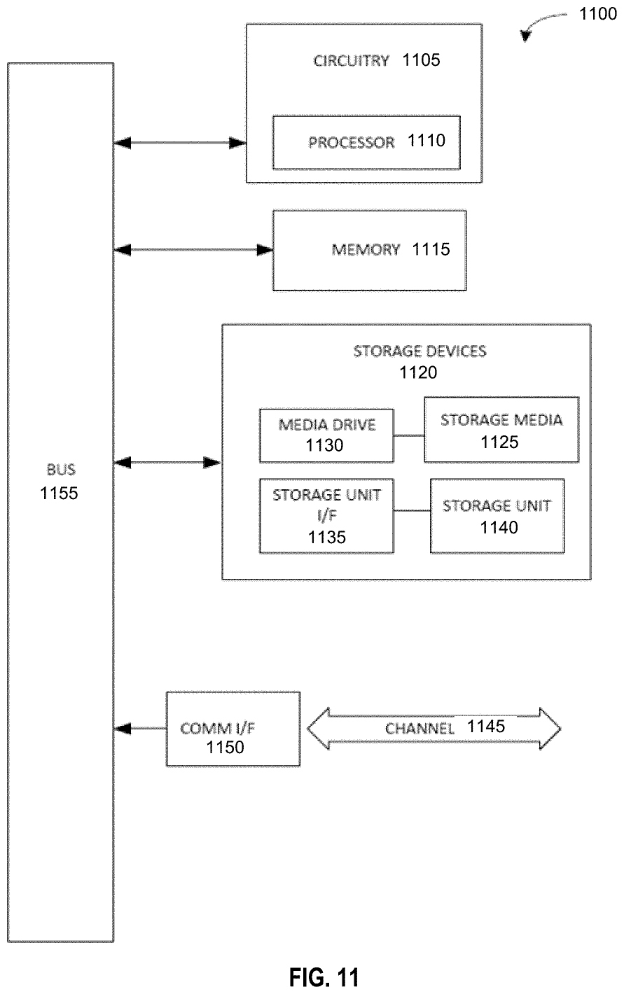

FIG. 11 illustrates example computing component 1100, which may in some instances include a processor on a computer system (e.g., control circuit). Computing component 1100 may be used to implement various features and/or functionality of embodiments of the systems, devices, and methods disclosed herein. With regard to the above-described embodiments set forth herein in the context of systems, devices, and methods described with reference to FIGS. 1-10, including embodiments involving the control circuit, one of skill in the art will appreciate additional variations and details regarding the functionality of these embodiments that may be carried out by computing component 1100. In this connection, it will also be appreciated by one of skill in the art upon studying the present disclosure that features and aspects of the various embodiments (e.g., systems) described herein may be implemented with respected to other embodiments (e.g., methods) described herein without departing from the spirit of the disclosure.

As used herein, the term component may describe a given unit of functionality that may be performed in accordance with one or more embodiments of the present application. As used herein, a component may be implemented utilizing any form of hardware, software, or a combination thereof. For example, one or more processors, controllers, ASICs, PLAs, PALs, CPLDs, FPGAs, logical components, software routines, or other mechanisms may be implemented to make up a component. In implementation, the various components described herein may be implemented as discrete components or the functions and features described may be shared in part or in total among one or more components. In other words, as would be apparent to one of ordinary skill in the art after reading this description, the various features and functionality described herein may be implemented in any given application and may be implemented in one or more separate or shared components in various combinations and permutations. Even though various features or elements of functionality may be individually described or claimed as separate components, one of ordinary skill in the art will understand upon studying the present disclosure that these features and functionality may be shared among one or more common software and hardware elements, and such description shall not require or imply that separate hardware or software components are used to implement such features or functionality.

Where components or components of the application are implemented in whole or in part using software, in embodiments, these software elements may be implemented to operate with a computing or processing component capable of carrying out the functionality described with respect thereto. One such example computing component is shown in FIG. 1. Various embodiments are described in terms of example computing component 1100. After reading this description, it will become apparent to a person skilled in the relevant art how to implement example configurations described herein using other computing components or architectures.

Referring now to FIG. 11, computing component 1100 may represent, for example, computing or processing capabilities found within mainframes, supercomputers, workstations or servers; desktop, laptop, notebook, or tablet computers; hand-held computing devices (tablets, PDA's, smartphones, cell phones, palmtops, etc.); or the like, depending on the application and/or environment for which computing component 1100 is specifically purposed.

Computing component 1100 may include, for example, one or more processors, controllers, control components, or other processing devices, such as a processor 1110, and such as may be included in 1105. Processor 1110 may be implemented using a special-purpose processing engine such as, for example, a microprocessor, controller, or other control logic. In the illustrated example, processor 1110 is connected to bus 1155 by way of 1105, although any communication medium may be used to facilitate interaction with other components of computing component 1100 or to communicate externally.

Computing component 1100 may also include one or more memory components, simply referred to herein as main memory 1115. For example, random access memory (RAM) or other dynamic memory may be used for storing information and instructions to be executed by processor 1110 or 1105. Main memory 1115 may also be used for storing temporary variables or other intermediate information during execution of instructions to be executed by processor 1110 or 1105. Computing component 1100 may likewise include a read only memory (ROM) or other static storage device coupled to bus 1155 for storing static information and instructions for processor 1110 or 1105.

Computing component 1100 may also include one or more various forms of information storage devices 1120, which may include, for example, media drive 1130 and storage unit interface 1135. Media drive 1130 may include a drive or other mechanism to support fixed or removable storage media 1125. For example, a hard disk drive, a floppy disk drive, a magnetic tape drive, an optical disk drive, a CD or DVD drive (R or RW), or other removable or fixed media drive may be provided. Accordingly, removable storage media 1125 may include, for example, a hard disk, a floppy disk, magnetic tape, cartridge, optical disk, a CD or DVD, or other fixed or removable medium that is read by, written to or accessed by media drive 1130. As these examples illustrate, removable storage media 1125 may include a computer usable storage medium having stored therein computer software or data.

In alternative embodiments, information storage devices 1120 may include other similar instrumentalities for allowing computer programs or other instructions or data to be loaded into computing component 1100. Such instrumentalities may include, for example, fixed or removable storage unit 140 and storage unit interface 1135. Examples of such removable storage units 140 and storage unit interfaces 1135 may include a program cartridge and cartridge interface, a removable memory (for example, a flash memory or other removable memory component) and memory slot, a PCMCIA slot and card, and other fixed or removable storage units 1140 and storage unit interfaces 1135 that allow software and data to be transferred from removable storage unit 840 to computing component 1100.

Computing component 1100 may also include a communications interface 1150. Communications interface 1150 may be used to allow software and data to be transferred between computing component 1100 and external devices. Examples of communications interface 150 include a modem or softmodem, a network interface (such as an Ethernet, network interface card, WiMedia, IEEE 802.XX, or other interface), a communications port (such as for example, a USB port, IR port, RS232 port Bluetooth® interface, or other port), or other communications interface. Software and data transferred via communications interface 1150 may typically be carried on signals, which may be electronic, electromagnetic (which includes optical) or other signals capable of being exchanged by a given communications interface 1150. These signals may be provided to/from communications interface 1150 via channel 1145. Channel 1145 may carry signals and may be implemented using a wired or wireless communication medium. Some nonlimiting examples of channel 1145 include a phone line, a cellular or other radio link, an RF link, an optical link, a network interface, a local or wide area network, and other wired or wireless communications channels.

In this document, the terms “computer program medium” and “computer usable medium” are used to generally refer to transitory or non-transitory media such as, for example, main memory 1115, storage unit interface 1135, removable storage media 1125, and channel 1145. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions may enable the computing component 1100 or a processor to perform features or functions of the present application as discussed herein.

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that can be included in the disclosed technology. The disclosed technology is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the technology disclosed herein. Also, a multitude of different constituent component names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “component” does not imply that the components or functionality described or claimed as part of the component are all configured in a common package. Indeed, any or all of the various components of a component, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A computer-implemented method comprising:
obtaining, from a catheter-based electrochemical sensor, drug concentration data associated with a first drug in a patient;
predicting, based on the first drug's drug concentration data, future pharmacokinetic parameters associated the first drug in the patient; and
providing, based on the first drug's predicted pharmacokinetic parameters, a first medical-related notification to a clinician.

2. The computer-implemented method of claim 1, wherein the drug concentration data comprises real-time drug concentration data associated with the first drug in the patient.

3. The computer-implemented method of claim 1, wherein predicting the first drug's future pharmacokinetic parameters comprises using Bayesian statistics to predict the first drug's future pharmacokinetic parameters.

4. The computer-implemented method of claim 1, further comprising:
constructing a dataset for the patient comprising the first drug's obtained drug concentration data; and
predicting, based on the patient's dataset, the first drug's future pharmacokinetic parameters and their likelihoods.

5. The computer-implemented method of claim 4, wherein the patient's dataset further comprises at least one of the following:
demographic information of the patient; and
monitored physiological data of the patient.

6. The computer-implemented method of claim 1, wherein the first drug's predicted pharmacokinetic parameters comprise parameters associated with the concentration of the first drug in the patient's plasma.

7. The computer-implemented method of claim 1, wherein the first medical-related notification comprises a recommendation to adjust infusion of the first drug.

8. The computer-implemented method of claim 1, further comprising:
obtaining, from the catheter-based electrochemical sensor, drug concentration data associated with a second drug in the patient;
predicting, based on the second drug's obtained drug concentration data, future pharmacokinetic parameters associated with the second drug in the patient; and
adjusting, based on the second drug's predicted pharmacokinetic parameters, administration of the second drug to the patient.

9. The computer-implemented method of claim 8, wherein the first drug comprises propofol and the second drug comprises fentanyl.

10. A system, comprising:
a catheter-based electrochemical sensor;
a processor; and
a memory configured to store instructions that, when executed by the processor, cause the processor to:
obtain, from the catheter-based electrochemical sensor, drug concentration data associated with a first drug in the patient;
predict, based on the first drug's real-time drug concentration data, future pharmacokinetic parameters associated with the first drug in the patient; and
adjust, based on the first drug's predicted pharmacokinetic parameters, administration of the first drug to the patient.

11. The system of claim 10, wherein the first drug's predicted pharmacokinetic parameters comprise parameters associated with the concentration of the first drug in the patient's plasma.

12. The system of claim 10, wherein predicting the first drug's future pharmacokinetic parameters comprises using Bayesian statistics to predict the first drug's future pharmacokinetic parameters.

13. The system of claim 10, wherein the stored instructions further comprise instructions to:
obtain, from the catheter-based electrochemical sensor, drug concentration data associated with a second drug in the patient;
predict, based on the second drug's drug concentration data, future pharmacokinetic parameters associated with the second drug in the patient; and
adjust, based on the second drug's predicted pharmacokinetic parameters, administration of the second drug to the patient.

14. The system of claim 12, wherein the catheter-based electrochemical sensor comprises:
a catheter tube; and
disposed within the catheter tube:
a first working electrode and a first reference electrode for detecting the first drug; and
a second working electrode and a second reference electrode for detecting the second drug.

15. The system of claim 13, wherein at least one of the first and second working electrode comprise a carbon paste material.

16. The system of claim 14, wherein the carbon paste material comprises a carbon nano tube-incorporated carbon paste.

17. The system of claim 14, wherein at least one of the first and second working electrode is coated with a polyvinyl chloride (PVC) material.

18. The system of claim 14, wherein at least one of the first and second working electrode is coated with multiple material layers, the multiple material layers comprising:
a PVC material layer;
an electrochemically reduced graphene oxide (erGO) material layer; and
a gold (Au) nanoparticle material layer.

19. The system of claim 14, wherein the first drug comprises propofol and the second drug comprises fentanyl.

20. A non-transitory computer-readable storage medium including instructions that, when executed by at least one processor, cause the processor to perform a method comprising:
obtaining, from a catheter-based electrochemical sensor inserted in a patient, real-time drug concentration data associated with a drug in the patient;
generating, from the drug concentration data, a patient-specific pharmacokinetic model;
using the patient-specific pharmacokinetic model to predict future pharmacokinetic parameters associated with the drug in the patient; and
providing, based on the predicted pharmacokinetic parameters, a medical-related notification to a clinician.
